(19) United States
(12) Patent Application Publication
(10) Pub. No.: US 2024/0271068 A1
(43) Pub. Date: Aug. 15, 2024

(54) A PETRI DISH

(71) Applicant: SUNITA SANJAY DANGE, BARAMATI (IN)

(72) Inventors: SUNITA SANJAY DANGE, BARAMATI (IN); SANJAY RAMCHANDRA DANGE, SATARA (IN); REKHA MOHAN GULVE, BEED (IN)

(21) Appl. No.: 18/567,710

(22) PCT Filed: Jul. 28, 2021
(86) PCT No.: PCT/IB2021/056860

§ 371 Date: Dec. 6, 2023

(30) Foreign Application Priority Data
Jun. 7, 2021 (IN) 202121025318

(51) Int. Cl. C12M 1/22; C12M 1/00; C12M 1/34; C12M 3/00

(52) U.S. Cl. CPC C12M 23/10; C12M 23/22; C12M 23/38; C12M 23/48; C12M 41/36; C12M 41/44

(57) ABSTRACT

A petri dish is provided. The petri dish includes a base plate configured to hold a material. The base plate includes a graduate scale with marking. The base plate also includes a plurality of sector lines arranged to divide the base plate into a plurality of sections. The petri dish also includes a top plate configured to be operable with the base plate; a detachable absorbent pad ring operatively coupled to a rim of the top plate. The detachable absorbent pad ring is configured to absorb moisture from the material within the petri dish. The petri dish also includes a magnifying glass of a predefined size housed on an inner surface of the top plate. The magnifying glass is configured to provide an enlarged view of the material. The magnifying glass operates when the top plate is closed on top of the base plate.

### CROSS-REFERENCE TO RELATED APPLICATION

This application claims priority from a Patent application filed in India having Patent Application No. 202121025318, filed on Jun. 7, 2021, and titled “A PETRI DISH” and a PCT Application No. PCT/IB2021/056860 filed on Jul. 28, 2021, and titled “A PETRI DISH.”

### FIELD OF INVENTION

Embodiments of a present disclosure relate to a petri dish, and more particularly to, a petri dish with gradient scale.

### BACKGROUND

A petri dish or a petri dish is a shallow transparent lidded dish that biologists use to hold growth medium in which cells can be cultured, originally, cells of bacteria, fungi and small mosses. Mostly in research and development laboratories of pharmaceutical industries, fermentation industries, medical or pathological laboratories, biotechnology and microbiology laboratories, or the like; plate assay techniques are used in day-to-day practice. In this assay strength of a compound is estimated from zone of inhibition or zone of exhibition or in case of biosurfactant, oil-displacement assay. In most of the approaches of using such assay or the petri dishes, a user must use a ruler or a scale to take measurements and note. In addition, taking bacterial count is a common practical in all above said fields, through some conventional approaches, differentiating the colonies and observing the growth is a tedious process and is not very accurate. Furthermore, in biosurfactant assay, as a test oil is layered on distilled water surface and a drop of biosurfactant to be tested is added, according to the strength of the biosurfactant oil is displaced. This is a quick response, and a bit more time taken in adjusting ruler scale leads to misinterpretation, also making the approach slower. These limitations make the system less reliable and less efficient. Furthermore, taking colony count from the Petri dish is one more practice, however, it becomes critical while counting hundreds of colonies from backside of the plate, in order to ease the process, a plate is usually divided into four quadrants manually. In addition, whenever a plate is filled up with about 20 ml of medium with approximation, some plates may get more, and some may get less medium. Observation of colony morphology is very basic requirement which makes the thing time consuming with unaided eyes. Also, collection of moisture in a lid poses a difficult task for a smooth handling of the Petri dish as well as in observations of results. Even sometimes this moisture interferes with results, thereby making the conventional approach less reliable and less efficient.

Hence, there is a need for an improved petri dish to address the aforementioned issue/s.

### BRIEF DESCRIPTION

In accordance with one embodiment of the disclosure, a petri dish is provided. The petri dish includes a base plate configured to hold a material. The base plate includes a graduate scale with marking. The base plate also includes a plurality of sector lines arranged to divide the base plate into a plurality of sections. The petri dish also includes a top plate configured to be operable with the base plate. The petri dish also includes a detachable absorbent pad ring operatively coupled to a rim of the top plate. The detachable absorbent pad ring is configured to absorb moisture from the material within the petri dish. The petri dish also includes a magnifying glass have a predefined size housed on an inner surface of the top plate. The magnifying glass is configured to provide an enlarged view of the material placed with the base plate. The magnifying glass operates when the top plate is closed on top of the base plate.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a schematic representation of a petri dish in accordance with an embodiment of the present disclosure;

FIG. 2 is a schematic representation of a displacement zone in the culture dish of FIG. 1 in accordance with an embodiment of the present disclosure;

FIG. 3 is a schematic representation of a sectored culture dish of FIG. 1 in accordance with an embodiment of the present disclosure;

FIG. 4 is a schematic representation of an arial view of a base plate with a detachable absorbent pad ring of FIG. 1 in accordance with an embodiment of the present disclosure;

FIG. 5 is a schematic representation of a back view of a top plate with the detachable absorbent pad ring of FIG. 1 in accordance with an embodiment of the present disclosure; and

FIG. 6 is a schematic representation of a top view of a magnified view from a magnifying glass on a top plate of FIG. 1 in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a petri dish. The petri dish includes a base plate configured to hold a material. The base plate includes a graduate scale with marking. The base plate also includes a plurality of sector lines arranged to divide the base plate into a plurality of sections. The petri dish also includes a top plate configured to be operable with the base plate. The petri dish also includes a detachable absorbent pad ring operatively coupled to a rim of the top plate. The detachable absorbent pad ring is configured to absorb moisture from the material within the petri dish. The petri dish also includes a magnifying glass have a predefined size housed on an inner surface of the top plate. The magnifying glass is configured to provide an enlarged view of the material placed with the base plate. The magnifying glass operates when the top plate is closed on top of the base plate.

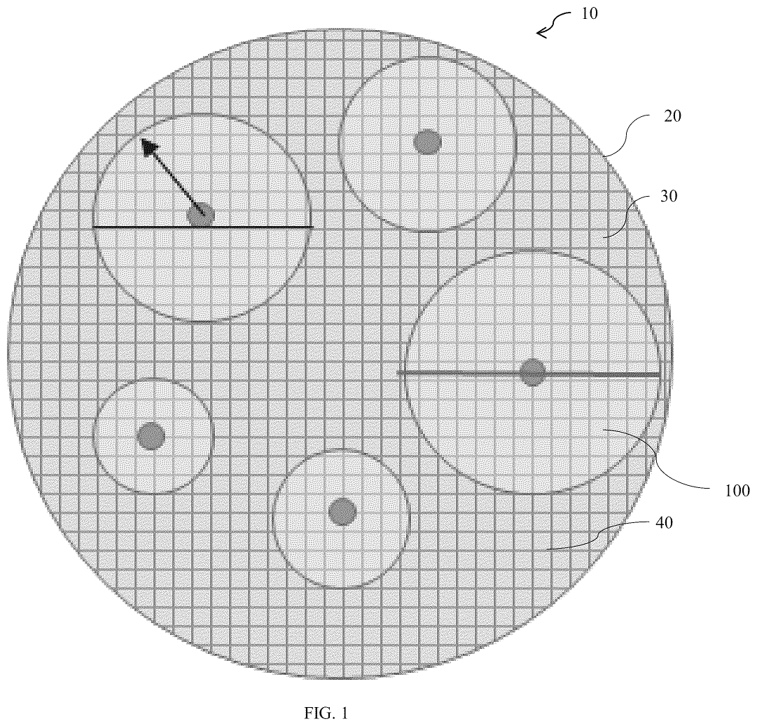

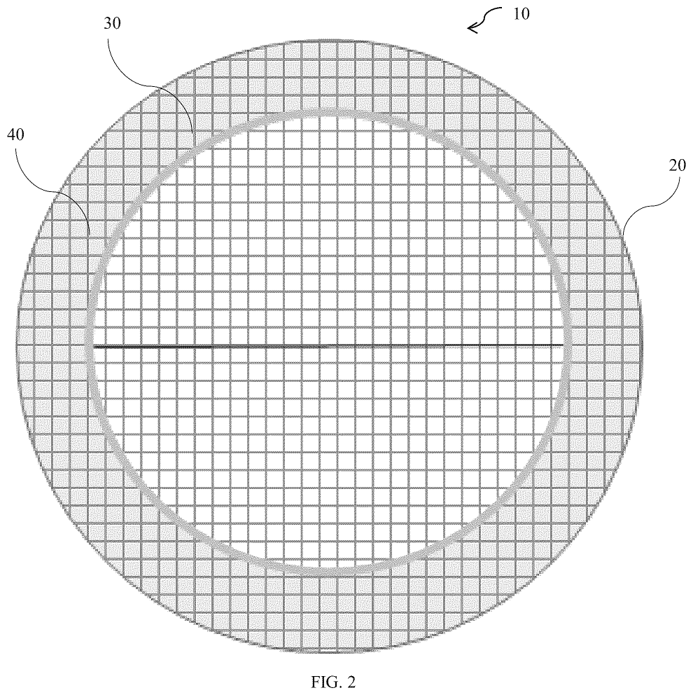

FIG. 1 is a schematic representation of a petri dish 10 in accordance with an embodiment of the present disclosure. The petri dish 10 includes a base plate 20 configured to hold a material. In one embodiment, the material may be one of a solid material, liquid material, plasma material, or a combination thereof. The base plate 20 includes a graduate scale 30 (as shown in FIG. 2) with marking. In one embodiment, the marking on the graduate scale 30 may be ruler markings of predefined dimensions, where is the marking is used to measure one or more zones of the base plate 20. In such embodiment, the pre-defined measurements may be corresponding to standard readings of a ruler or a scale which may be used in mathematical applications. In one embodiment, the one or more zones may be at least one of zone of inhibition, zone of stimulation, zone of exhibition, zone of displacement, or a combination thereof.

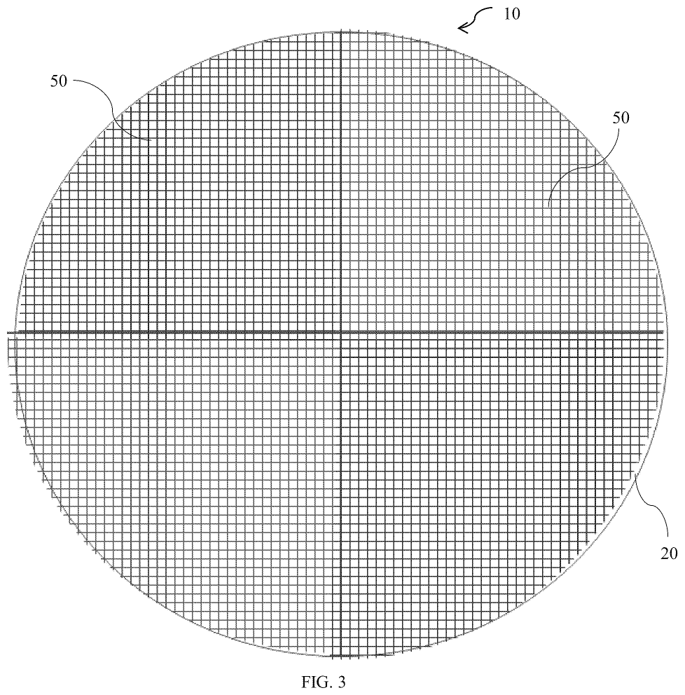

The base plate 20 also includes a plurality of sector lines 40 arranged to divide the base plate 20 into a plurality of sections 50 (as shown in FIG. 3). In one embodiment, the plurality of sections 50 may be one or more sectors as per graph standard used in the mathematical applications. In some embodiment, the plurality of sections 50 may be configured to enable a user to differentiate one or more colonies formed by the material within the base plate 20.

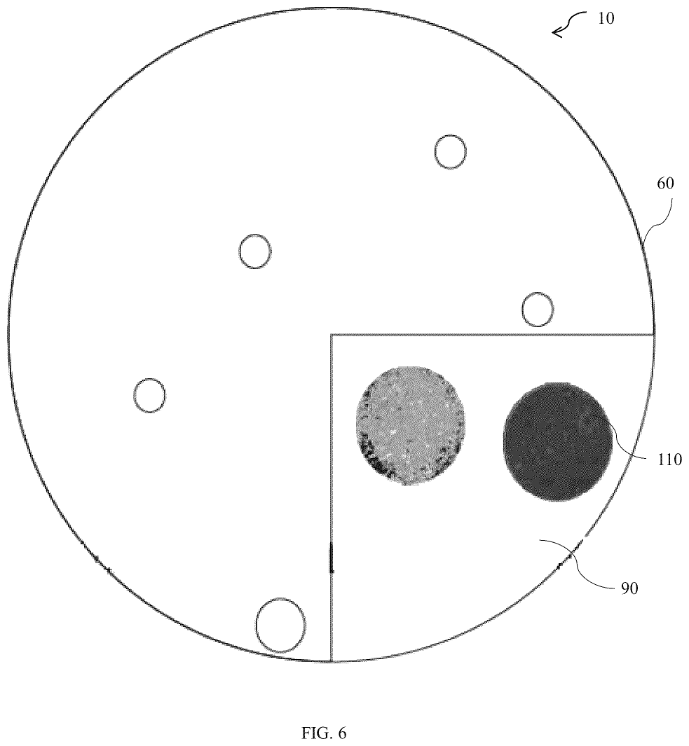

Furthermore, the petri dish 10 includes a top plate 60 (as shown in FIG. 6) configured to be operable with the base plate 70. In one embodiment, a radius of the top plate 60 may be slightly greater than a radius of the base plate 20. Because of a radius difference, the top plate 60 may be made to sit on an outer rim of the base plate 20.

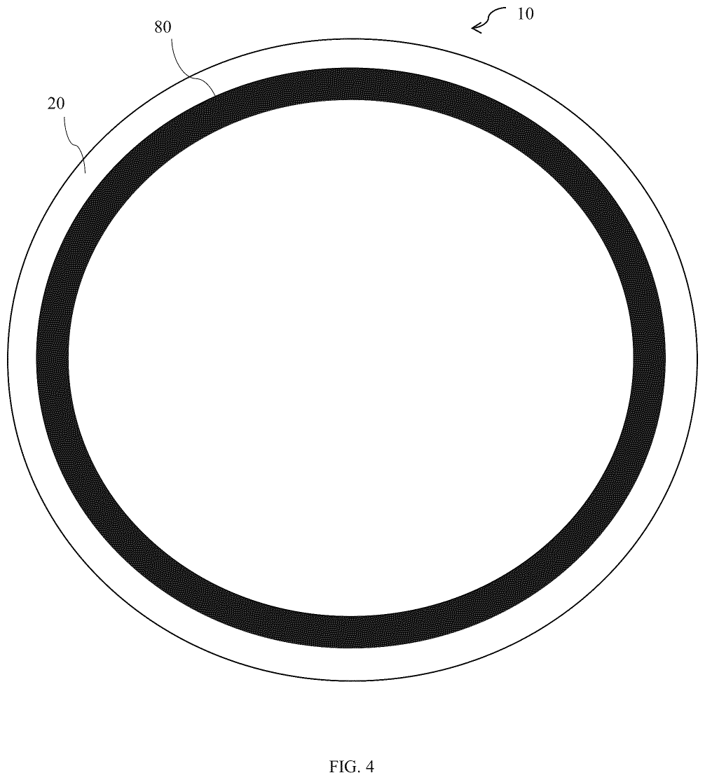

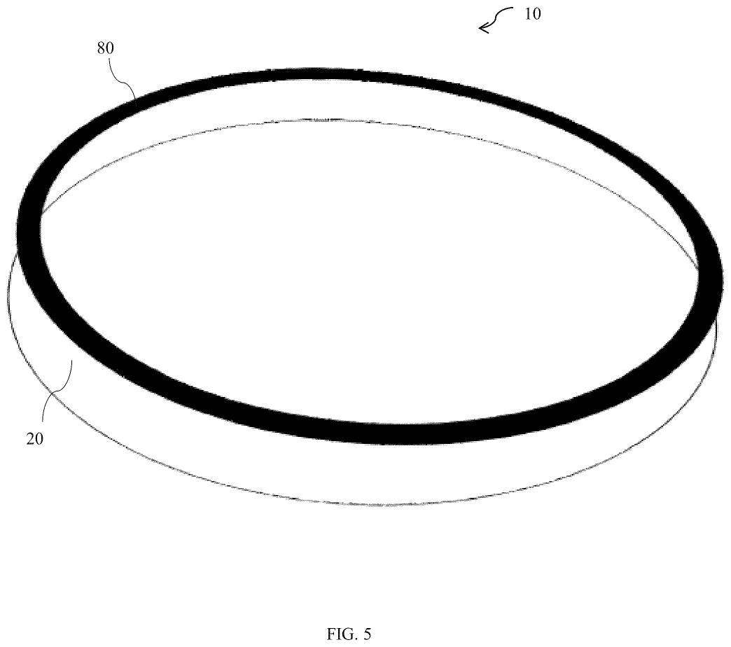

The petri dish 10 also includes a detachable absorbent pad ring 80 (as shown in FIG. 4 and FIG. 5) operatively coupled to a rim of the top plate 60. The detachable absorbent pad ring 80 is configured to absorb moisture from the material within the petri dish 10. In one embodiment the material when getting cultured may release an amount of moisture content which may be collected within the petri dish 10 due to the tight packaging of the top plate 60 with the base plate 20. The detachable absorbent pad ring 80 may be placed at the rim of an inner wall of the top plate 60 which helps in absorbing this excess moisture content within the petri dish 10. The detachable absorbent pad ring 80 may be detached from the top plate 60 when not required.

The petri dish 10 further includes a magnifying glass 90 (as shown in FIG. 9) have a predefined size housed on an inner surface of the top plate 60. The magnifying glass 90 is configured to provide an enlarged view of the material placed with the base plate 20. The magnifying glass 90 operates when the top plate 60 is closed on top of the base plate 20. In one embodiment, the top plate 60 may include a plurality of magnifying glasses which may be attached to either the inner surface of the top plate 60 or to an outer surface of the top plate 60. In one specific embodiment, a part of the top plate 60 may be fabricated with the magnifying glass material which may eliminate a two layer of top plate 60 and the magnifying glass 90 attached. In such embodiment, the magnifying glass acts at the top plate 60 for that corresponding region where the top plate 60 material may be replaced. In one specific embodiment, the top plate 60 may include magnifying glass of a quarter size of the top plate 60 which is placed at one of the quarters off the top plate 60.

When the top plate is rotated along its circumference, the magnifying glass 90 can be used to view the material inside the bottom plate 20. In another specific embodiment, the top plate 60 mean include at least two magnifying glasses 90, wherein each of the at least two magnifying glasses may be of a dimension equal to the quarter size of the top plate 60. The at least two magnifying glasses 90 may be placed opposite to each other leaving the other quadrants to be composed of a normal material of that of the top plate 60. In such embodiment, two quadrants off the bottom plate 20 can be viewed simultaneously. Upon rotation of the top plate 60, the other two quadrants of the bottom plate 20 can be viewed. The magnifying glass 90 may be used to view the growth of the material in the bottom plate 20 from the top plate 110. The material may have formed a plurality of colonies 100 (as shown in FIG. 1), which can be measured using the graduate scale 30 and the plurality of sector lines 40.

In one exemplary embodiment, the petri dish 10 may further include a screw and screw cap operatively coupled to the base plate 20 and the top plate 60 respectively. The screw and the screw cap are configured to enable the operation of the top plate 60. The operation of the top plate 60 may include opening and closing of the top plate from the base plate 20. More specifically, in order to hold and align the top plate 60 with the base plate 20, the screw and the screw cap are being used, when the screw cap is attached to the top plate 60, and the screw to the bottom plate 20. On rotating the top plate 60 in a pre-defined direction at a pre-defined angle, the top plate 60 gets either detached or attached from the bottom plate 20. Further, on rotating the top plate 60 in an opposite direction of the pre-defined direction, the operation of the top plate 60 in may be reversed.

In another exemplary embodiment, the petri dish 10 may further include one or more magnetic strips operatively coupled to an inner rim of the top plate 60 and an outer rim of the bottom plate 20. The one or more magnetic strips may be configured to operate the top plate 60. The operation of the top plate 60 may be opening and closing of the top plate from the base plate 20.

More specifically, the one or more magnetic strips may be attached on the inner rim of the top plate 60 and each of the one or more magnetic strips may be placed at every pre-defined distance from an adjacent magnetic strip. Further, the one or more magnetic strips are placed at an adjacent corresponding place on the inner rim of the top plate 60. In operation, an amount of force needs to be applied on the top plate 60 to detach the magnetic effect of one or more magnetic strips between the bottom plate 20 and the top plate 60.

In one specific embodiment, petri dish 10 may further include at least one marking line representing a level for the material to be filed within the petri dish, wherein the at least one marking line is placed on at least one side wall of the base plate 20. In such embodiment, the at least one marking line comprises a line for holding the material of about 20 ml in volume.

Various embodiments of the present disclosure enable the petri dish to provide a graduate scale to take the measurement of the material inside the bottom plate. Also due to the magnetic strips being used the petri dish can hold the bottom plate and the top plate in place without harming the material within the bottom plate. In addition the screw means used for the placement of the top plate get the bottom plate also helps in locking door petri dish without harming the material inside. in addition the magnifying glass is used helps the user to see the growth of the material or the functionality of the material within the petri dish without actually opening the top plate from the bottom plate, thereby keeping the material safe from the environmental conditions.

Also, the absorbent material used in the petri dish helps in absorbing the additional moisture content produced during the growth of the material inside the petri dish the absorbent material can be disposed after use. Unlike in the conventional approaches where the additional moisture content had to be separated from the petri dish by removing the top plate from the bottom plate which used to lead to the exposure of the material to the environmental conditions which can be overcome using the absorbent material are they absorbent strips. In cases where there is no requirement of the absorbent strips, the strips can be detached from the petri dish, thereby making the petri dish more reliable and efficient in terms of performance usage and protection of the material.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

I claim:

1. A petri dish comprising:
a base plate configured to hold a material, wherein the base plate comprises:
a graduate scale with marking; and
a plurality of sector lines arranged to divide the base plate into a plurality of sections;
a top plate configured to be operable with the base plate,
characterized in that,
a detachable absorbent pad ring operatively coupled to a rim of the top plate, and configured to absorb moisture from the material; and
a magnifying glass have a predefined size housed on an inner surface of the top plate, wherein the magnifying glass is configured to provide an enlarged view of the material placed with the base plate, wherein the magnifying glass operates when the top plate is closed on top of the base plate.

2. The petri dish as claimed in claim 1, wherein the marking on the graduate scale comprises ruler markings of predefined dimensions, where is the marking is used to measure one or more zones of the base plate.

3. The petri dish as claimed in claim 2, wherein the one or more zones comprises zone of inhibition, zone of stimulation, zone of exhibition, zone of displacement, or a combination thereof.

4. The petri dish as claimed in claim 1, wherein the plurality of sections is configured to enable a user to differentiate one or more colonies formed by the material within the base plate.

5. The petri dish as claimed in claim 1, comprising one or more magnetic strips operatively coupled to an inner rim of the top plate and an outer rim of the bottom plate, wearing the one or more magnetic strips is configured to operate the top plate, wherein the operation of the top plate comprises opening and closing of the top plate from the base plate.

6. The petri dish as claimed in claim 1, comprising a screw and screw cap operatively coupled to the base plate and the top plate respectively, wherein the screw and the screw cap is configured to enable the operation of the top plate, wherein the operation of the top plate comprises opening and closing of the top plate from the base plate.

7. The petri dish as claimed in claim 1, comprising at least one marking line representing a level for the material to be filed within the petri dish, wherein the at least one marking line is placed on at least one side wall of the base plate.

8. The petri dish as claimed in claim 7, wherein the at least one marking line comprises a line for holding the material of about 20 ml in volume.
